(19) United States
(12) Patent Application Publication
(10) Pub. No.: US 2018/0143279 A1
(43) Pub. Date: May 24, 2018

(54) METHOD, COMPUTER AND MAGNETIC RESONANCE APPARATUS FOR DETERMINATION OF PARAMETERS FOR THE MAGNETIC RESONANCE APPARATUS

(71) Applicant: Siemens Healthcare GmbH, Erlangen (DE)

(72) Inventor: Thomas Kluge, Hirschaid (DE)

(73) Assignee: Siemens Healthcare GmbH, Erlangen (DE)

(21) Appl. No.: 15/815,858

(22) Filed: Nov. 17, 2017

(30) Foreign Application Priority Data
Nov. 18, 2016 (DE) 102016222785.8

(51) Int. Cl. G01R 33/565; A61B 5/055

(52) U.S. Cl. CPC G01R 33/565; G06T 1/0007; A61B 5/055

(57) ABSTRACT

In a method and computer for automatic determination of parameters for an examination with a magnetic resonance apparatus, a modeling is determined that establishes a relationship between the parameters and relations. Each relation describes a mathematical relationship between the parameters of the respective relation. To this end, values or ranges of values of specific of the parameters are acquired, which are predetermined by a user of the magnetic resonance apparatus. The parameters are determined such that the relations are complied with.

### BACKGROUND OF THE INVENTION

### Field of the Invention

The present invention concerns the automatic determination of parameters of a magnetic resonance apparatus in order, on the basis of these parameters, to carry out an examination of an examination object with the magnetic resonance apparatus.

### Description of the Prior Art

Before a diagnostic examination with a magnetic resonance apparatus (scanner) can be carried out, a parameter set valid for this magnetic resonance scanner must first be created. In such cases the valid parameter set must comply with all limitations or system boundaries that are defined by the magnetic resonance scanner and the examination object (usually a human patient). Additionally, the parameter set should come as close as possible to the requirements of the technician or physician who is responsible for carrying out the examination, so that the result of the examination accords with the wishes of the technician or the supervising physician.

Because of the complexity of the measuring procedures in magnetic resonance tomography and because of the complexity of the interrelationships between the parameters to be set, there is no known process in the prior art that resolves the parameter selection problem comprehensively for all interrelations (user interface, sequences, hardware, patient) and does so correctly (i.e. all relations are valid for the parameter set selected or determined).

### SUMMARY OF THE INVENTION

An object of the present invention is to solve the aforementioned parameter selection problem automatically.

In accordance with the invention this object is achieved by a method for automatic determination of parameters for an examination with a magnetic resonance apparatus having the following steps executed in a computer:
Determining a modeling, which establishes relationships between the parameters and relations. In such cases each relation determines a mathematical relationship between the parameters of the respective relation.
Acquiring or reading in values or ranges of values of specific parameters, which are predetermined by a user of the magnetic resonance system. Accordingly, in this step, values or ranges of values are predetermined for specific parameters of the parameters to be determined for the examination with the magnetic resonance system.
Automatic determination of the parameters such that the relations of the modeling are complied with. In such cases particular attention is paid to the parameters being determined in such a way that the previously acquired values or ranges of values are complied with. In other words parameters of which the values are predetermined by the user will not be changed during automatic determination and for parameters for which the user has predetermined ranges of values, a value that lies within the respective range of values is determined in accordance with the invention.

Since the modeling includes all necessary mathematical relationships between the parameters, advantageously the parameters can be created automatically while taking account of particular specifications of the user. This thus enables the inventive method advantageously to resolve the parameter selection problem comprehensively and correctly. In such cases the modeling corresponds to a graph, wherein each node of the graph represents a relation or a parameter and wherein each edge of the graph connects a relation to a parameter. Each of the parameters is in particular represented by precisely one node. In this case each relation (i.e. each node that corresponds to a relation) is directly connected via an edge in each case to only those parameters of which the mathematical relationship describes the corresponding relation. In other words each edge connects a parameter with that relation, which describes a mathematical relationship with regard to this parameter.

On the basis of this graph it can be established very rapidly, for example, which relations influence a change of a parameter. This means that the graph makes it possible very rapidly for the relations or parameter relationships that have become invalid because of the parameter change to be corrected by further parameter changes, i.e. to be made valid once again. For their part, those relations, which will invalid as a result of the further parameter changes and are corrected by yet further parameter changes, etc., are determined on the basis of the graph. This possible inventive process will be described again below in detail.

In accordance with the invention the modeling or the graph can be constructed hierarchically. To do this, specific parameters and specific relations are combined in a suitable manner into part graphs (composite models), which are reusable. This means that the same part graph can be present at different points in the graph. In this case a part graph can also contain another part graph. The part graph forms a node in relation to the graph, so that each node of the graph can be a relation, a parameter or a part graph.

The use of part graphs advantageously facilitates the creation of the modeling.

Before the parameters are determined on the basis of the modeling, it is advantageous to break down all hierarchy levels and thus create a flat modeling. To do this all part graphs are resolved, so that the graph no longer contains any part graphs.

The mathematical relationships can be conservative in such cases. In this context a conservative relationship is understood as being that, when the relations describe conservative mathematical relationships, a parameter set that complies with the relations ensures that a measurement of an examination object by means of the magnetic resonance system can be carried out with the parameters determined in this way, without the system boundaries or safety requirements that relate to the magnetic resonance system or to the patient being violated.

Conservative mathematical relationships will take precedence over exact mathematical relationships, if these conservative relationships are easier to handle or simpler to formulate than the exact mathematical relationships. The conservative relationships or models virtually represent a type of agreement between the magnetic resonance system and the measurement method with which both sides have to comply. I.e when the conservative relationships or models described in the form of relations are fulfilled by the automatically determined parameter set, it is ensured that a measurement sequence carried out with this parameter set does not violate any limitations relating to the magnetic resonance system or the examination object.

The mathematical relationships or underlying models in particular involve equations or inequalities. Thus the modeling forms a system of equations and/or inequalities that the parameters must fulfill.

In such cases each relation can have a specification for each parameter for which a relationship is established by this relation, as to whether the respective relation can be resolved according to this parameter. When the relation involves an equation, the specification of the equation specifies for each parameter whether the equation can be solved in accordance with this parameter; i.e. whether the equation can be rearranged such that the corresponding parameter stands on its own on one side of the equal sign.

When a relation can be solved in accordance with a parameter, then this parameter can be determined very easily after a parameter change on the basis of the current (e.g. currently changed) values of the other parameters of the relation such that the relation is valid again, even with the changed parameters.

In accordance with the invention at least a few of the relations can also describe an image quality or a measure of an image quality as a function of other parameters. This image quality relates in this case to images that will be created on the basis of data acquired during the examination by means of the magnetic resonance system. Possible measures for the image quality are, for example:
The contrast of the images,
A signal-to-noise ratio of the data on the basis of which the images will be created,
A measure of the extent (e.g. number and scope) of artifacts within the images.

Because the image quality can also be computed on the basis of the relations, the image quality for the images that are created as a function of the parameters determined in accordance with the invention can advantageously be displayed to the user. The user can then make changes to the parameters, in order to improve the image quality. Moreover, in accordance with the invention, the parameters can also be determined automatically such that the image quality complies with at least one predetermined measure of quality. Accordingly, on the basis of the relations describing the image quality, better account can be taken of user requirements.

In accordance with a preferred inventive form of embodiment the inventive method comprises at least one specification or heuristic, which prescribes how the parameters are to be changed to comply with the relations. The at least one specification will then be used during the determination of the parameters.

When a parameter of a relation has been changed, then in most cases there are a number of combinations of values for the other parameters of the relation, so that the relation is valid again. These combinations of values in particular will be restricted by the specification in a way desired by the user, so that the automatically determined parameters correspond to the requirements of the user as well as possible.

This at least one specification can be application-dependent in this case. This means that the at least one specification can be dependent on the type of examination or measurement sequence that will be used.

Possible specifications in such cases are as follows:

The parameters are to be selected so that examination is as short as possible. In a measurement sequence the parameters of the measurement sequence are accordingly selected so that the time taken by the measurement sequence is as short as possible.

The parameters are to be selected so that the images created by the examination are as sharp as possible. In a measurement sequence the parameters of the measurement sequence are accordingly selected so that the images created by the measurement sequence have an image sharpness that is as high as possible.

A parameter is only to be changed in one direction. This means that each of the parameters will only be enlarged or reduced in accordance with this specification. Or to put it differently, once a parameter has been changed, it will not be changed back.

The parameters are to be selected so that the contrast of the images created by the examination lies where possible above a predetermined contrast threshold value. In a measurement sequence the parameters of the measurement sequence are accordingly selected so that the images created by the measurement sequence have a contrast that lies above the contrast threshold.

The parameters are to be selected so that the signal-to-noise ratio (SNR) of the images created by the examination lies where possible above a predetermined SNR threshold value. In a measurement sequence the parameters of the measurement sequence are accordingly selected so that the images created by the measurement sequence have an SNR that lies above the SNR threshold value.

The parameters are to be selected so that a measure for artifacts within the images created by the examination lies where possible below a predetermined artifact threshold value. In a measurement sequence the parameters of the measurement sequence are accordingly selected so that the images created by the measurement sequence only exhibit artifacts such that the measure for artifacts lies below the artifact threshold value.

In accordance with the invention the parameters can also be determined such that a compromise between specific specifications is achieved. For example a target function can be defined as a function of the overall measurement time, the image sharpness, the contrast, the signal-to-noise ratio and/or the measure of artifacts. The parameters can then be determined so that this target function exhibits an optimum. This advantageously enables the parameters to be determined so that the image quality is better than a specific threshold value (e.g. contrast threshold value, SNR threshold value, artifact threshold value) and that in addition the overall measurement time is as short as possible.

Above and beyond this, in accordance with the invention, there is the possibility for at least one of the relations to comprise a specification, which prescribes, for one or for a number of parameters of the relation, how the corresponding parameter is to be changed as a function of a change of another of the parameters of the relation.

This specification, for example, prescribes for a relation that a parameter of the relation is to be reduced if another parameter of the relation is enlarged. This specification can accordingly speed up the renewed fulfillment of a relation for a change of a parameter of the relation.

In accordance with the invention the specification or heuristic can be embodied programmable and able to be exchanged by software means.

It is precisely the exchangeability of the specification that makes possible a simple adaptation of the determination of the parameters to any given objectives.

In a further embodiment, a time for carrying out the determination of the parameters is measured. When this carrying-out time exceeds a predetermined period of time the determination of the parameters is interrupted in order to obtain assistance from the user.

When the automatic method for determination of the parameters has still not found a solution after the predetermined period of time (i.e. no parameter set could yet be found that fulfills all relations), support from or assistance by the user is requested. In order to support users themselves when they are entering their assistance, the modeling can be graphically presented to the user.

The parameters to be determined in particular involve the parameters of a measurement sequence of the magnetic resonance system.

In a further embodiment, the method includes the following steps:
Checking which of the relations will be violated by the (current) parameters.
Changing the (current) parameters of those relations that were recognized in the preceding step as violated. Subsequently the method returns to the previously described step of checking, until the checking step no longer discovers any violated relation (or until the carrying-out time exceeds the previously described period of time).

In a further embodiment, a target function can be defined, which computes a target value as a function of a predetermined set of the parameters to be determined. The determination of the parameters can then be undertaken on the basis of an optimizer, so that on the one hand the relations will be complied with and on the other hand the target value will be optimized. In other words the optimizer searches through all valid parameter sets for that parameter set at which the target value (depending on definition of the target function) has its maximum or minimum value (i.e. its global optimum). For example this enables a valid parameter set to be determined for which the overall measurement time is (verifiably) minimal.

The present invention also encompasses a computer for determination of parameters for an examination with a magnetic resonance apparatus. The computer has a control processor with which a modeling is determined, which establishes relationships between the parameters and relations. In such cases each relation describes a mathematical relationship between the parameters of the respective relation. This modeling, once determined, is stored in a memory. Via an input interface, values or ranges of values of specific parameters are entered by a user. The control processor determines the parameters such that the relations are complied with.

The advantages of the inventive computer essentially correspond to the advantages of the inventive method, which have described above in detail.

The present invention also encompasses a magnetic resonance apparatus that includes the inventive computer.

The present invention also encompasses a non-transitory, computer-readable data storage medium encoded with programming instructions that, when loaded into a computer, cause the computer to execute any or all of the embodiments of the method according to the invention, as described above.

The software can be source code (e.g. C++), which still has to be complied (translated) and linked or which only has to be interpreted, or can involve executable software code, which, to be executed, then just has to be loaded into the corresponding processing unit or control device.

The electronically-readable data medium, can be a DVD, a magnetic tape, a hard disk or a USB stick, on which electronically-readable control information, in particular software is stored.

The present invention advantageously virtually represents a compromise between an unstructured sequence-specific and developer-specific source code and a parameter determination that determines the parameters based on models with an equation solver (solver or optimizer). Compared to the former, the present invention advantageously allows a large degree of automation. Compared to the latter, in accordance with the invention, any given complex mathematical relations (not only linear or convex) can be used, without this resulting in processing time problems, as is frequently the case with solvers in accordance with the prior art.

In accordance with the invention it is possible to create a model from the graph, with which a solver can create a valid parameter set. This is possible when the relations of the graph have a mathematical structure that is suitable for the creation of a valid parameter set with the solver. In this case it is also possible to create an optimum parameter set in relation to a target function.

The present invention advantageously represents a pragmatic, generally valid solution approach for the determination of parameters for an examination with a magnetic resonance system. The parameter set determined as a rule, although it does not involve the optimum parameter set, advantageously makes sure that this parameter set complies with the relations and thus with the system restrictions described therein. As has already been described above, in accordance with the invention, an optimum parameter set in relation to a target function can also be created, in that a model is created from the graph, with which an equation solver (solver or optimizer) can create a valid parameter set.

The inventive method is based on a formal description of a measurement sequence by means of mathematical models (relations). The determination of a valid parameter set as a function of the modeling can be carried out generically and independent of the measurement method just used or of the measurement sequence just used. In other words the software, which determines a valid parameter set using modeling as its starting point, can be independent of the software, which determines the modeling. This also enables a mathematical optimizer to be used for example in order to determine the valid parameter set, using the modeling as its starting point.

The inventive method advantageously does not require any “blind testing out” of the parameter set, as is known currently under the term binary search for example and is used according to the prior art for creation of a parameter set. Therefore, in accordance with the invention, by comparison with the prior art, significant savings can be made in processing power, since the conflicts arising in the determination of parameters will be resolved in other ways.

Provided all known restrictions and limitations of the hardware and of the examination object are taken into consideration in the modeling, downstream checks, which are carried out for example with the aid of a representative test run of the measurement sequence, can advantageously be avoided. This means that advantageously an interruption of a measurement already started because implementations have exceeded a given limit also no longer occurs.

The present invention is based on a formal mathematical model in the form of the graph. This enables the model to be used at system level (i.e. at the system level or by the MR system itself or systematically), in order to determine the parameters automatically.

In accordance with the invention user-related application parameters, hardware-related measurement parameters and limitations of the magnetic resonance system and of the examination object can be simplified and modularized by the inventive modeling.

The customer receives an inventive facility or an inventive magnetic resonance system that behaves consistently and creates the parameters of a measurement sequence with minimum interaction with the user or operator.

In accordance with the invention the modeling can also be used to define the requirements of a future magnetic resonance system as a function of specific application parameters.

The modeling can be undertaken with SysML (Systems Modeling Language), Modelica (an object-oriented modeling language for physical models) or a domain-specific language created separately for the present purpose.

### BRIEF DESCRIPTION OF THE DRAWINGS

In FIG. 1 schematically illustrates an inventive magnetic resonance apparatus.

In FIG. 2 shows basic parameters of an example of a measurement sequence of the inventive magnetic resonance apparatus.

In FIG. 3 shows an inventive modeling in the form of a graph.

FIG. 4 is a flowchart of the inventive method.

### DESCRIPTION OF THE PREFERRED EMBODIMENTS

FIG. 1 shows a schematic illustration of the inventive magnetic resonance apparatus 5. The magnetic resonance apparatus 5 has a scanner 3, with which the magnetic field necessary for the MR examination is created in a measurement space 4, a table or support board 2, a control computer 6, with which the scanner 3 is controlled and MR data are acquired from the scanner 3, and a computer 20 connected to the control computer 6.

The control computer 6 has an activation processor 11, a reception processor 12 and an evaluation processor 13. During the creation of an image dataset MR data are acquired by the scanner 3 from the reception processor 12, wherein the scanner 3 and the table 2 are activated by the activation processor 11 such that MR data in a measurement volume 15, which is located inside the body of a patient O lying on the table 2, are acquired.

The evaluation processor 13 then prepares the MR data such that the data can be displayed graphically on a screen 8 of a terminal 7 of the computer 20 and such that MR images created in accordance with the invention are displayed. As well as the graphic display of MR data, specifications for determination of the parameters for the measurement sequence can be specified by the user for the magnetic resonance apparatus 5 with the terminal 7, which as well as the screen 8, has a keyboard 9 and a mouse 10. As well as the terminal 7, the computer 20 has control processor 16 and a memory 17. The software for the control computer 6 can also be loaded into the control computer 6 via the terminal 7. This software of control computer 6 can also include the inventive method in this case. It is also possible in this case for the inventive method to be contained in software that runs in the control processor 16. Regardless of the software in which the inventive method is contained, the software can be stored on a DVD 14, so that this software can then be read by the computer 20 from the DVD 14 and either copied into the control processor 6 or into the memory 17 of the computer 20 itself.

FIG. 2 shows a measurement sequence 24 with the most important parameters for a magnetic resonance apparatus.

The measurement sequence 24 shown in FIG. 2 involves a comparison between an exemplary and greatly simplified measurement sequence and measurement sequences used in practice. Structurally any given sequence types and any given limitations can be presented in this way in accordance with the invention.

The gradient echo measurement sequence 24 shown emits an RF excitation pulse 26 with the amplitude RFA during a period of time tS. A slice selection gradient GS is applied at the same time as the RF excitation pulse 26. After the RF excitation pulse 26, during the period of time tP, the gradient GSre, to cancel the phase response arising during the excitation, and the gradient GP for impressing a phase response are applied. The gradient GApre serves to pre-phase the readout gradient GA. Subsequently MR data is read out during the period of time tA, while the readout gradient with the amplitude GA is applied. For half the readout time tA the gradient echo is produced, wherein the distance in time between the time of the maximum of the RF excitation pulse 26 and the half of the readout time tA corresponds to the echo time TE.

FIG. 3 shows an inventive modeling in the form of a graph. In this figure the nodes of the graph 25 on the one hand comprise parameters, namely input parameters 21, constant parameters 22 and other parameters 23, and on the other hand relations 1.

The relations mapped as an example in the graph 25 of FIG. 3 will be described in detail below.

$$calcRFA \text{:} RFA = \frac{RFConst \cdot BTP}{tS}$$

According to relation or equation calcRFA, the amplitude RFA of the RF excitation pulse 26 corresponds to the quotient of the product of RFConst and BTP (a product of bandwidth and duration of the RF excitation pulse and thereby a possible measure for the quality of the slice profile) and the duration tS of the RF excitation pulse 26.

$$calcMS \text{:} MS = \frac{BTP}{γ \cdot dS}$$

According to relation or equation calcMS, the slice selection gradient moment MS corresponds to the quotient of BTP and the product of the gyromagnetic ratio γ and the slice depth dS.

$$calcMSre \text{:} MSre = \frac{MS}{2}$$

According to relation or equation calcMSre, MSre corresponds to half of the slice selection gradient moment MS.

$$calcMP \text{:} MP = \frac{1}{γ \cdot dY}$$

According to relation or equation calcMP, the gradient moment MP of the gradient GP corresponds in the y direction to the reciprocal of the product of the gyromagnetic ratio γ with the resolution dY in the y direction.

$$calcMAPre \text{:} MAPre = \frac{MA}{2}$$

According to relation or equation calcMApre, MAPre corresponds to half the readout gradient moment MA (i.e. of the gradient moment of the gradient GA in the x direction).

$$calcMA \text{:} MA = \frac{1}{γ \cdot dX}$$

According to relation or equation calcMA, the readout gradient moment MA corresponds to the reciprocal of the product of the gyromagnetic ratio γ with the resolution dX in readout direction or x direction.

$$calcTE \text{:} TE = \frac{tS}{2} + tP + \frac{tA}{2}$$

According to relation or equation calcTE, the echo time TE corresponds to the sum of half of the RF excitation pulse duration tS plus the period of time tP between RF excitation and beginning of the readout plus half of the readout time tA.

calcTR: TR=tS+tP+tA

According to relation or equation calcTR, the repetition time TR corresponds to the sum of the RF excitation pulse duration tS plus the period of time tP between RF excitation and beginning of the readout plus the readout time tA.

calcMO: Mx=Gx·tx

According to relation or equation calcMO, a gradient moment (e.g. MS, MSre,

MP, MApre, MA) corresponds to the product of a gradient (e.g. GS, GSre, GP, GApre, GA) and the period of time (e.g. tS, tP, tA) during which the gradient is present.

checkRFmax: RFA≤RFAmax

According to relation or inequation calcRFmax, the amplitude of the RF excitation pulse may never exceed the maximum possible amplitude RFAmax of the RF excitation pulse.

checkGmax: Gx≤Gmax

According to relation or inequation calcRFmax, a gradient (e.g. GS, GSre, GP, GApre, GA) may never exceed the maximum possible gradient strength Gmax.

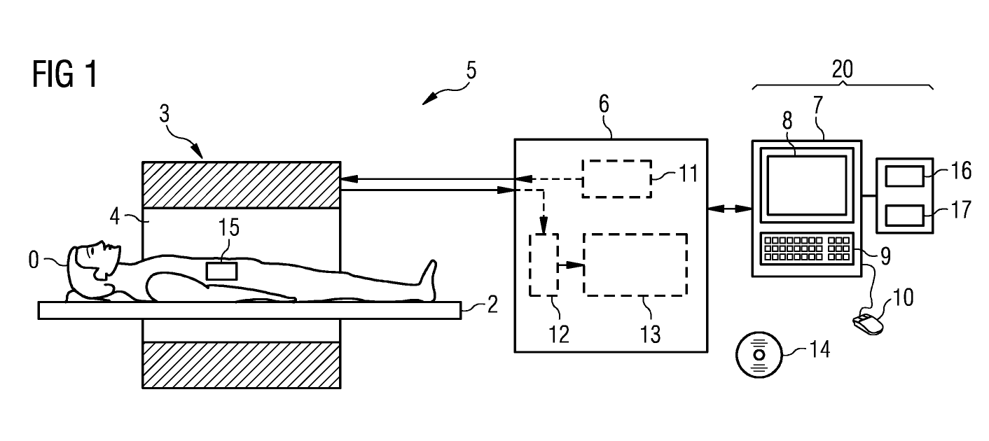

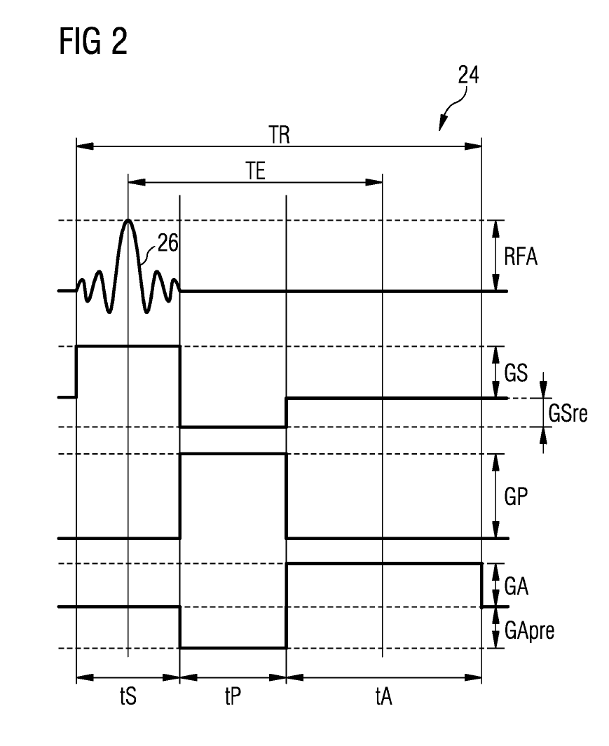

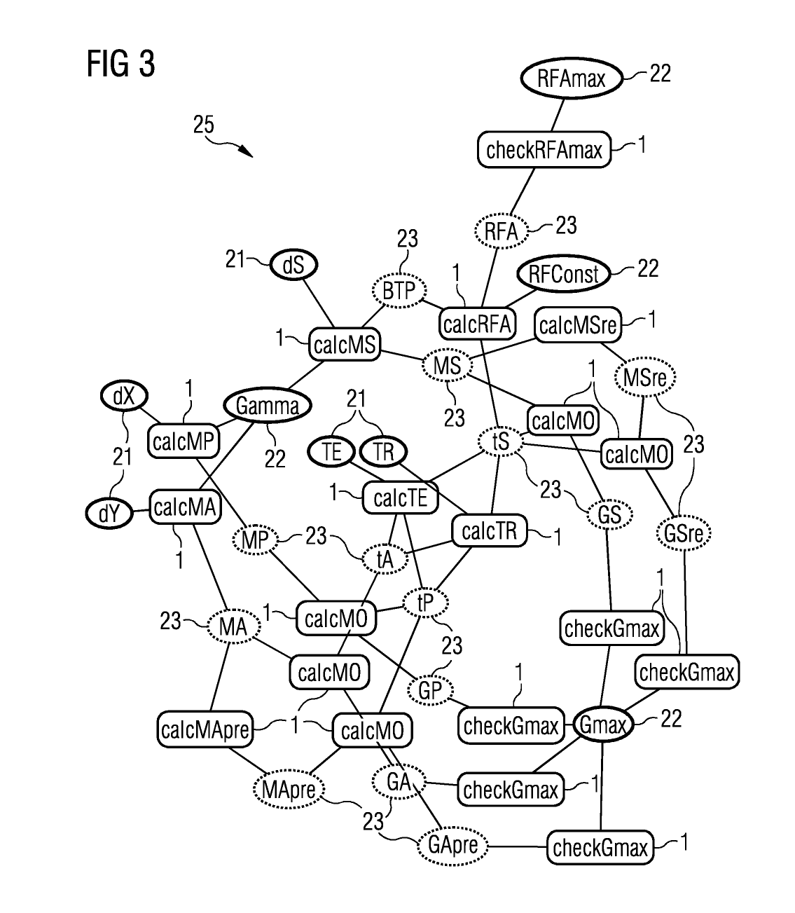

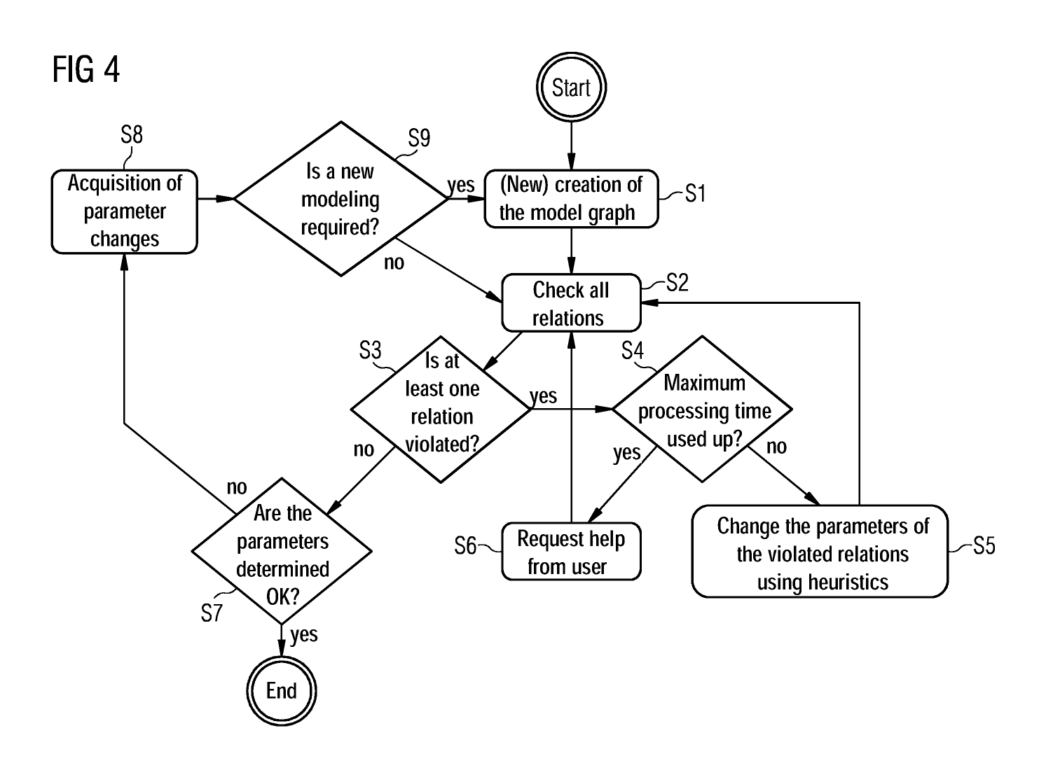

FIG. 4 shows the flowchart of the inventive method. In step S 1, starting from particular specifications of a user, a modeling, for example the graph 25 shown in FIG. 3, is determined. Subsequently, in step S2, all relations 1 of the modeling 25 are checked. I.e. a check is made as to whether the current parameter set, which was determined with the inventive method, fulfills the previously described relations 1. If it is recognized in step S3 that no relation 1 has been violated, the inventive method branches to step S7. In step S7 the user checks whether the parameters determined by the inventive method are in accordance with their wishes. If they are, the inventive method is ended. The parameter set determined in accordance with the invention can be used to acquire MR data of an examination object with a measurement sequence with the magnetic resonance system.

If the user is not in agreement with the parameters determined, in step S8 parameter changes are acquired. If in step S9 it is recognized that because of the changed parameters a new modeling or a new model graph 25 is required, the inventive method branches to step S1, in which this new modeling is determined (i.e. the inventive method starts again so to speak). If no new modeling is required, the inventive method branches to the already described step S2. In this case it can arise that user parameters or the changes to user parameters change the structure of the graph. In this case a branch is made to step S1, in which the model graph of the modeling is reconstructed.

If it is recognized in step S3 that at least one relation is violated by the current parameters, the inventive method branches to step S4. In this step S4 a check is made as to whether the current time needed to carry out the inventive method has already exceeded a threshold value (i.e. whether the maximum computing time for determination of the parameters is used up). If the carrying-out time has exceeded the threshold value, the inventive method branches to step S6, in which the user is asked for help. The user can change specific parameters in this step S6, in order, by doing so, to make possible the determination of a parameter set that fulfills all relations. After step S6 the inventive method continues at the already described step S2.

If it is recognized in step S4 that the carrying-out time has not yet exceeded the threshold value, the inventive method continues at step S5. In this step S5 the parameters of those relations that have been recognized in the checking in step S2 as violated relations are changed. For the change of the parameters heuristics are used, which in their turn can depend on predetermined strategies or on user specifications. After step S5, the inventive method continues at the already described step S2. As an alternative the step S5 (under the previously described conditions and with the restrictions mentioned above) can be carried out by a mathematical optimizer or equation solver.

The steps described in conjunction with FIGS. 2 to 4 can be carried out by the control computer 6 or the control processor 16. To do this the computer 6 or processor 16 can access program modules or instructions provided in a memory that, when executed by the computer 6 or the processor 16, put the steps described above into effect.

Although modifications and changes may be suggested by those skilled in the art, it is the intention of the Applicant to embody within the patent warranted hereon all changes and modifications as reasonably and properly come within the scope of the Applicant's contribution to the art.

1. A method for automatic determination of parameters for conducting an examination in a magnetic resonance (MR) data acquisition scanner of an MR apparatus, said method comprising:
in a computer, determining a modeling that establishes a relationship between said parameters defined by relations, each relation describing a mathematical relationship between parameters of the respective relation;
entering values or ranges of values of specific ones of said parameters into said computer that are predetermined by an operator of the MR apparatus;
in said computer, determining final values of said parameters that cause said relations to be complied with; and
making the final parameters available from the computer in electronic form.

2. A method as claimed in claim 1 comprising determining said modeling as a graph having a plurality of nodes, with each node of the graph representing one of said relations or one of said parameters, and with each edge of the graph connecting one of said relations to one of said parameters, and wherein each of said relations is connected only with an edge directly to parameters that the mathematical relationship of the respective relation describes.

3. A method as claimed in claim 2 comprising determining said modeling to be hierarchical by said relationships and said parameters being combined into a portion of said graph, with each node of the graph representing one of said relationships, one of said parameters, or said portion of said graph.

4. A method as claimed in claim 3 wherein said modeling comprises hierarchy levels and, before determining said parameters, breaking down all of said hierarchy levels by resolving all portions of said graph in order to make said modeling flat.

5. A method as claimed in claim 1 comprising employing conservative mathematical relationships to determine said model, said conservative mathematical relationships causing said parameters that satisfy said relations to ensure that examination of a subject by operating said MR data acquisition scanner with said parameters can be implemented without violating system boundaries or safety specifications.

6. A method as claimed in claim 1 comprising selecting said mathematical relationships from the group consisting of equations and inequalities.

7. A method as claimed in claim 1 wherein at least some of said relations describe an image quality of images created by an examination conducted with said MR data acquisition scanner operated with said parameters.

8. A method as claimed in claim 1 comprising using at least one specification to determine said parameters that describes how said parameters can be changed in order to comply with said relations.

9. A method as claimed in claim 8 comprising selecting said specifications from the group consisting of requiring the parameters to be selected in order to make an examination with said parameters as short as possible, selecting said parameters in order to require that images obtained with said parameters are as sharp as possible, changing said parameters only in one direction of said modeling;
selecting said parameters in order to make an image contrast of an image obtained with said parameters above a predetermined contrast threshold value, selecting said parameters in order to make a signal-to-noise ratio of measurement data acquired by operating the MR data acquisition scanner with said parameters above a predetermined signal-to-noise ratio threshold value, and selecting said parameters in order to make a measure of artifacts in an image below a predetermined artifact threshold value.

10. A method as claimed in claim 1 comprising determining said parameters according to a specification for at least one of said relations that specifies how said at least one parameter is to be changed dependent on a change to another of said parameters of said at least one relation.

11. A method as claimed in claim 1 comprising determining said parameters according to a specification that is programmable in said computer and exchangeable by a software modification.

12. A method as claimed in claim 1 comprising:
measuring an implementation time that accumulates during the determination of the parameters in said computer; and
interrupting the determination of the parameters in said computer if said time exceeds a predetermined period of time.

13. A method as claimed in claim 1 comprising using parameters of a measurement sequence of said MR data acquisition scanner, as said parameters to be determined.

14. A method as claimed in claim 1 comprising:
in said computer, checking whether said relations are violated and changing said parameters of relations that are identified as being violated, and repeating said check after making said change.

15. A method as claimed in claim 1 comprising:
defining a target function in said computer that determines a target value dependent on a value of at least one of said parameters; and
determining said parameters so as to have respective values that optimize said target function.

16. A computer for automatic determination of parameters for conducting an examination in a magnetic resonance (MR) data acquisition scanner of an MR apparatus, said computer comprising:
a processor configured to determine a modeling that establishes a relationship between said parameters defined by relations, each relation describing a mathematical relationship between parameters of the respective relation;
an input interface in communication with said processor that receives values or ranges of values of specific ones of said parameters into said processor that are predetermined by an operator of the MR apparatus;
said processor being configured to determine final values of said parameters that cause said relations to be complied with; and
an output interface in communication with said processor via which said processor is configured to make the final parameters available from the processor in electronic form.

17. A magnetic resonance (MR) apparatus comprising:
an MR data acquisition scanner;
a processor configured to determine a modeling that establishes a relationship between said parameters defined by relations, each relation describing a mathematical relationship between parameters of the respective relation;
an input interface in communication with said processor that receives values or ranges of values of specific ones of said parameters into said processor that are predetermined by an operator of the MR apparatus;
said processor being configured to determine final values of said parameters that cause said relations to be complied with; and
an output interface in communication with said processor via which said processor is configured to provide the final parameters from the processor in electronic form to said MR data acquisition scanner.

18. A non-transitory, computer-readable data storage medium encoded with programming instructions, said storage medium being loaded into a computer of a magnetic resonance (MR) apparatus that comprises an MR data acquisition scanner, said programming instructions causing said computer to:
determine a modeling that establishes a relationship between said parameters defined by relations, each relation describing a mathematical relationship between parameters of the respective relation;
receive values or ranges of values of specific ones of said parameters into said computer that are predetermined by an operator of the MR apparatus;
determine final values of said parameters that cause said relations to be complied with; and
make the final parameters available from the computer in electronic form.
